(19) United States
(12) Patent Application Publication
(10) Pub. No.: US 2001/0051133 A1
(43) Pub. Date: Dec. 13, 2001

(54) MERCY HEALING FROM GOD INTRAVENOUS HUMAN IMMUNODEFIENCY VIRUS (HIV) DETERGENT

(76) Inventor: CHEVELLE R. MOORE (JONES-MOORE), UTICA, NY (US)

Correspondence Address: DAVID M. GIGLIO, ATTORNEY AT LAW, 231 ELIZABETH STREET, UTICA, NY, 13501, US

(21) Appl. No.: 09/459,102

(22) Filed: Dec. 13, 1999

(51) Int. Cl. A61K 9/00; A61K 7/06; A61K 33/02

(52) U.S. Cl. 424/43; 424/719; 424/74

(57) ABSTRACT

The Viral Detergent consisting of the intravenous dissemination of pure distilled water carbonated at the highest level of calibration both prepared and stored at a consistent temperature (between 33°-35° Fahrenheit) will allow for the complete eradication of Human Immunodeficiency Virus (HIV) in subjects treated, and will preserve human life through the mercy of God.

### BACKGROUND OF THE INVENTION

“In God We Trust” is written all over our American currency. I believe God is asking us to trust Him in the area of healing people from Human Immunodefieciency Virus (HIV) and Acquired Immunodeficiency Syndrome (AIDS). I believe that God has revealed to me the means by which to rid the human body of HIV and in its more advance state AIDS. I believe that God has revealed this healing to demonstrate His continued love and mercy for those that believe on Him, and to demonstrate His existence to them that don't know Him as God.

No money from this invention shall be used on a personal basis by me or any man. The resources from this invention shall be placed into an established foundation created to retrieve lost souls into the Kingdom of God. While you may consider this unbelievable and absurd, this is okay. In God's Word it states: “Beloved believe not every spirit, but try the spirits whether they are of God: because many false prophets are gone out into the world [I John 4:1].”

Following the exact directions explained in this patent will without neither flaw nor negative side effects allow individuals to be ridden of HIV in their blood stream and further prevent the progression of AIDS within the human body. The God I serve is not a half God. He created the world and has the ability to do all things completely and perfectly. There is no hit or miss with God. Either He revealed this healing to me or I missed Him. I believe God's word “For the scripture saith, whosoever believeth on him [God] shall not be ashamed [Romans 10:11].”

### BRIEF SUMMARY OF THE INVENTION

Human Immunodeficiency Virus (HIV) and Acquired Immunodeficiency Syndrome (AIDS) can now be ridden from the human body by use of the Mercy Healing From God: Viral Detergent. This Viral Detergent is a natural chemical composition that is to be administered directly into the blood stream via intravenous dissemination under strictly specified controlled environmental conditions. Upon administering the Viral Detergent as treatment the HIV will die while in the blood stream and will be eliminated from the body through normal metabolic processes via the excretory system.

### DETAILED DESCRIPTION OF THE INVENTION

Human Immunodeficiency Virus (HIV) and Acquired Immunodeficiency Syndrome (AIDS) can now be ridden from the human body by use of the Mercy Healing From God: Viral Detergent.

Viral Detergent: An intravenous dissemination of pure distilled water carbonated at the highest level of carbonation, prepared and stored at a consistent temperature of 33°-35° Fahrenheit, and administered within the same temperature range. The volume of Viral Detergent administered is contingent upon the weight-capacity of the subject receiving treatment.

Note: Have each new intravenous bag ([IV) drip/leak freely for 5 minutes before connecting the IV to the patient. This allows for the release of extraneous air build-up,

Treatment using Viral Detergent is administered at the following volume levels, time intervals and ages.

Volume Levels/Time/Temperature+External Treatment Factor

100 cc/100 lbs/hour

500 cc/50 lbs/hour

250 cc/25 lbs/hour

100 cc/24 lbs >/hour

Temperature ranges of the administered Viral Detergent should be established based on the progression of the disease. If the subject receiving treatment is in the HIV stage, the temperature at which the Viral Detergent is administered should be at 35° Fahrenheit. If the Subject receiving treatment has progressed into AIDS, the temperature which the Viral Detergent is administered should be 33°-34° Fahrenheit.

External Treatment Factor: Room temperature during the administration of the Viral Detergent is an important variable to the success of treatment, and reduces the possibility of aneurysm. The subject receiving treatment needs to be prepped for the intravenous

### DETAILED DESCRIPTION OF THE INVENTION Continued

dissemination of the Viral Detergent. Patient preparation includes: (a) anaesthetic or sedative (tolerance for pain); and (b) being placed in a room of 50° Fahrenheit for 30 minutes prior to administering the Viral Detergent.

### Example Subjects

Subject 1 [210 lbs HIV]=2100 cc 3 hours at 35° F.

Subject 2 [141 lbs AIDS]=1500 cc 2 hours at 33°-34° F.

Subjects weighing less than 100 lbs are to incorporate age as a treatment factor with subjects age 20 and older.

Subject 3 [93 lbs HIV] 20+years=850 cc 1 hours at 35° F.

Subject 4 [93 lbs HIV] 19−years=850 cc 2 hours 35° F.

Note: Any subject under 1 year of age should not be treated by this means. Any subject actively in a pneumonias stage should not be treated until the stage passes. If it is determined by a medical team that the pneumonias stage will not pass as due to AIDS, then the Viral Detergent as a treatment regiment can be used, but at the lowest level of intensity (lower than the otherwise recommended volume, at the highest possible temperature). At the signs of progression for this subject (i.e.,recovery of pneumonia) then treatment regiment can be administered or re-administered as described above for subjects not presently experiencing pneumonia.

1. I claim that: the investigative and the medical use of the Viral Detergent as a treatment regimen will prove to generate desirable levels in suppressing Human Immunodeficiency Virus (HIV) ranges 100%, with no return, unless the subject is re-infected with the HIV.

2. I claim that: the investigative and the medical use of the Viral Detergent in individuals in the advance stages of AIDS, will allow individuals to recover, not returning to a positive HIV state, unless the subject is later re-infected with the HIV Virus.
